(19) United States
(12) Patent Application Publication
(10) Pub. No.: US 2002/0197278 A1
(43) Pub. Date: Dec. 26, 2002

(54) Covalent coupling of botulinum toxin with polyethylene glycol

(75) Inventor: Anthony Allison, Belmont, CA (US)

Correspondence Address: SWANSON & BRATSCHUN L.L.C., 1745 SHEA CENTER DRIVE, SUITE 330, HIGHLANDS RANCH, CO, 80129, US

(73) Assignee: SURROMED, INC., MOUNTAIN VIEW, CA

(21) Appl. No.: 10/176,957

(22) Filed: Jun. 21, 2002

Related U.S. Application Data
(60) Provisional application No. 60/299,807, filed on Jun. 21, 2001.

(51) Int. Cl. A61K 39/08; C07K 14/33; A61K 39/02

(52) U.S. Cl. 424/239.1; 530/410; 424/236.1

(57) ABSTRACT

Modified toxins including botulinum toxin or tetanus toxin coupled to polyethylene glycol, pharmaceutical compositions of modified toxins, and methods for their use are provided. The methods include treating inappropriate muscle contraction, and treatments for cosmetic purposes.

### CROSS-REFERENCE TO RELATED APPLICATIONS

This application claims the benefit of U.S. Provisional Application No. 60/299,807, entitled “Covalent Coupling of Botulinum Toxin with Polyethylene Glycol,” filed on Jun. 21, 2001.

### FIELD OF THE INVENTION

The present invention improves the efficacy of botulinum toxin for the treatment of disorders associated with inappropriate muscle contraction and for cosmetic applications. The toxin is modified so as to decrease its side effects and prolong its clinical utility.

### BACKGROUND OF THE INVENTION

The neurotoxins produced by the bacterium Clostridium botulinum exert their paralytic effect at the neuromuscular junction by preventing the release of acetylcholine. Seven serologically distinct botulinum toxins, designated A through G, have been characterized, as well as tetanus toxin. These toxins have similar molecular weights (about 150 kDa) and subunit structures, as well as sequence homologies. The toxins comprise a short peptide chain of about 50 kDa which is considered to be responsible for the toxic properties, and a larger peptide chain of about 100 kDa which is considered to be necessary to enable attachment and penetration of the presynaptic membrane. The short and long chains are linked together by means of disulfide bridges. Although the target proteins differ, all botulinum toxins are believed to exert their neuroparalytic effects by the same mechanism, suppression of acetylcholine release from nerve terminals (reviewed by Brin, M. F. Botulinum toxin: chemistry, pharmacology, toxicology, and immunology. Muscle and Nerve, Supplement 6:S146-168, 1997, and the references cited therein, incorporated herein by reference).

Botulinum toxins A and B are approved for use by regulatory authorities in many countries for the treatment of cervical dystonia. They have also been used for the treatment of other disorders involving inappropriate muscle contraction, including intractable low back pain, cerebral palsy, spastic paresis, blepharospasm, hyperhydrosis, hypersialorrhoea, and whiplash, migration and tension headaches. Botulinum toxins have also been administered to reduce deep facial wrinkles and for other cosmetic applications (Carruthers A. and Carruthers, J. Clinical indications and injection technique for the cosmetic use of botulinum A exotoxin. Dermatol. Surg. 24:1189-1194, 1998; Carruthers et al., U.S. Pat. No. 6,358,917, issued Mar. 19, 2002, both incorporated herein by reference).

Botulinum toxins are typically injected into the target site, and it is desirable to limit the action of the toxin to that site. Botulinum toxin can spread through muscle fascia by diffusion (Shaari, C. et al. Quantifying the spread of botulinum toxin through muscle fascia. Laryngoscope 101:960-964, 1991, incorporated herein by reference). Frequently effects on nearby muscles are demonstrable by electromyography (Buchman, A. S. et al. Quantitative electromyographic analysis of changes in muscle activity following botulinum therapy for cervical dystonia. Clin. Neuropharm. 16:205-210, 1993, incorporated herein by reference). This can result in undesirable side effects, for example vertical strabismus and ptosis associated with treatment of blepharospasm, and spread of the toxin to pharyngeal and laryngeal muscles when the target muscles are in the neck (see Shaari et al.). Electromyographic studies show effects of botulinum toxin even on distant muscles (Erdal, J. et al. Long-term botulinum toxin treatment of cervical dystonia—EMG changes in injected and noninjected muscles. Clin. Neurophysiol. 110:1650-1654, 1999, incorporated herein by reference). Significant atrophy of type IIB muscle fibers has been observed in leg muscles after repeated injection of botulinum toxin for cervical dystonia (Ansred, T. et al. Muscle fiber atrophy in leg muscles after botulinum toxin type A treatment of cervical dystonia. Neurology 48:1440-1442, 1997, incorporated herein by reference). Systemic effects include malaise and delayed emptying of the gallbladder (Schneider, P. et al. Gallbladder dysfunction induced by botulinum A toxin. Lancet 342:811-812, 1993, incorporated herein by reference). Rare complications of botulinum toxin administration include urinary incontinence, dysphagia and a generalized botulismlike syndrome (Boyd, R. N. et al. Transient urinary incontinence after botulinum A toxin. Lancet 348:481-482, 1997; Truite, P. J., Lang, A. E. Severe and prolonged dysphagia complicating botulinum toxin A injections for dystonia in Machado-Joseph disease. Neurology 46:846, 1996; Bakheit, A. M. et al. Generalized botulism-like syndrome after intramuscular injections of botulinum toxin A: a report of two cases. J. Neurol. Neurosurg. Psychiatry 62:198, 1997, all of which are incorporated herein by reference).

The action of botulinum toxin on nerve terminals is irreversible, but axon sprouting reverses the clinical effects, usually in two to six months. Injection of the toxin must then be repeated. The development of resistance to botulinum toxin is an important clinical problem. Antibodies against the toxin are presumed to be responsible for most cases of resistance. Naumann, M. et al. Depletion of neutralising antibodies resensitises a secondary non-responder to botulinum A neurotoxin. J. Neurol. Neurosurg. Psychiatry 65:924-927, 1998; Hauna, P. A. et al. Comparison of the mouse protection assay and an immunoprecipitation assay for botulinum toxin antibodies. J. Neurol. Neurosurg. Psychiatry 66:612-616, 1998, incorporated herein by reference. It is therefore also desirable to reduce the immunogenicity of the toxin.

### SUMMARY OF THE INVENTION

The present invention provides a method for treating disorders of inappropriate muscle contraction by administering a botulinum toxin covalently coupled to polyethylene glycol. Pegylation of the toxin is site directed so that it does not interfere with the neuroparalytic effect of the toxin but reduces its immunogenicity. Preferred proteins for pegylation are botulinum toxins A or B, because there is substantial clinical experience of their use. However another botulinum toxin (C through G) or tetanus toxin may also be pegylated and administered to patients. Pegylation of botulinum toxin will increase its molecular weight and decrease its diffusion from the injection site, thereby reducing side effects. The reduced immunogenicity of pegylated toxin will decrease the development of resistance.

### DETAILED DESCRIPTION OF THE INVENTION

To prepare botulinum toxin, Clostridium botulinum is cultured in a fermenter, acidified and harvested by centrifugation. The precipitated crude toxin is solubilized and purified using standardized methods ensuring quality and sterility (Schantz, E. J., Johnson, E. A. Properties and use of botulinum toxins and other microbial neurotoxins in medicine. Microbiol. Rev. 56:80-99, 1992, incorporated herein by reference). The preferred toxins for pegylation are botulinum toxin A or B, since there is already much information on their clinical use. However, another botulinum toxin (C through G) or tetanus toxin may also be modified and used according to the invention.

Information about the mechanism of action and three-dimensional structure of botulinum toxins is known (Lacy, D. B. et al. Crystal structure of botulinum neurotoxin type A and implications for toxicity. Nat. Struct. Biol. 5:898-902, 1998, incorporated herein by reference; Brin, supra), as well as the definition of major immunogenic determinants (Bavari S. et al. Identifying the principal protective antigenic determinants of type A botulinum toxin. Vaccine 16:1850-1856, 1998, incorporated herein by reference). This information is important in the selection of the sites for pegylation.

The site-specific pegylation is carried out by methods well-known in the art (Veronese, F. M. Peptide and protein PEGylation: a review of problems and solutions. Biomaterials 22:405-417, 2001, incorporated herein by reference). PEG is attached to botulinum toxin at a site, or sites, so that it retains the capacity to prevent acetylcholine release from nerve terminals. Furthermore, PEG is preferably attached onto or close to a sequence of amino acids defining a major immunogenic epitope. See Bavari S. et al., supra. For example, PEG may be attached to the carboxyl or amino terminals of proteins or to ε-amino groups of lysine residues. PEG can also be attached selectively to the sulfhydryl groups of naturally occurring or introduced cysteine residues. However, in view of the role of disulfide bonding between heavy and light chains during the rearrangement of the botulinum toxin molecule, this strategy must be used with caution so as not to interfere with its activity. Again, these examples of site-specific pegylation are illustrative but not comprehensive.

Included in the invention are botulinum toxins that are genetically modified so as to facilitate site-specific pegylation. Site-directed mutagenesis is carried out by methods well-known in the art. For example, site-directed mutagenesis may be used to replace selectively arginine codons (see Hershfield, M. S. et al. Use of site-directed mutagenesis to enhance the epitope-shielding effect of covalent modification of proteins with polyethylene glycol. Proc. Natl. Acad. Sci. U.S.A. 88:7185-7189, 1991, incorporated herein by reference). The additional ε-amino group of lysine provides a convenient attachment site that can be introduced into a region of the protein that is highly immunogenic. Another example is site-directed mutagenesis to introduce a cysteine residue at a specific location which is immunogenic and far from the active site of a protein (He, X.-H. et al., supra). These examples are intended to be illustrative and not comprehensive.

The pegylated botulinum toxin is formulated, stored and assayed for potency under standardized conditions (see Schantz and Johnson, supra). It is then tested for immunogenicity in mice and/or other experimental animals. Pegylation has been shown to suppress the immunogenicity of therapeutically used proteins, including arginase (Savoca, K. V. et al. Preparation of a non-immunogenic arginase by the covalent attachment of polyethylene glycol. Biochim. Biophys. Acta 578:47-53, 1979, incorporated herein by reference), purine nucleoside phosphorylase (Hershfield, M. S. et al. Use of site-directed mutagenesis to enhance the epitope-shielding effect of covalent modification of proteins with polyethylene glycol. New Engl. J. Med. 310:589-596, 1987, incorporated herein by reference), and interleukin-2 (Katre, N.V. Immunogenicity of recombinant IL-2 modified by covalent attachment of polyethylene glycol. J. Immunol. 144: 209-213, 1990, incorporated herein by reference). Pegylation has also been used experimentally to reduce the immunogenicity of a chimeric toxin (Wang, Q.-C. et al, Polyethylene glycol-modified chimeric toxin composed of transforming growth factor oc and Pseudomonas exotoxin. Cancer Res. 53: 4588-4594, 1993, incorporated herein by reference).

The advantages of using other pegylated proteins in humans are well known. In patients with chronic hepatitis C, a regimen of pegylated interferon alfa-2a given once a week is more effective than a regimen of the same interferon given three times weekly (Zeuzem, S. et al. Peginterferon alfa-2a in patients with chronic hepatitis C. New Engl. J. Med. 343:1666-1672, 2000, incorporated herein by reference). Pegylated megakaryocyte growth and development factor reduces the duration of thrombocytopenia following cancer chemotherapy (Hofmann, W. K. et al. Megakaryocyte growth factors: is there a new approach for management of thrombocytopenia in patients with malignancies? Leukemia 13:14-18, 1999, incorporated herein by reference).

Increasing the molecular weight of proteins by pegylation can also influence their pharmacokinetics and prolong in vivo efficacy (Clark, R. et al. Long-acting growth hormones produced by conjugation with polyethylene glycol. J. Biol. Chem. 271:21969-21977, 1996, incorporated herein by reference). The resistance of pegylated proteins to proteolysis may also contribute to the prolongation of their half-life in the body (references in Xe, X.-H. et al. Reducing the immunogenicity and improving the in vivo activity of trichosanthin by site-directed pegylation. Life Sciences 65:355-368, 1999, incorporated herein by reference).

In the case of botulinum toxins it is desirable to increase the molecular weight of the molecule to reduce its diffusion from the site of injection. This can be achieved by coupling several molecules of PEG to one molecule of toxin or by enlarging the size of the PEG covalently attached to the toxin. Electromyography and histological assessment can be used to assess the diffusion of the toxin from the injection site (Borodic, G. E. Histologic assessment of dose related diffusion of muscle fiber response after therapeutic botulinum A toxin injections. Mov. Disord 9:31-39, 1994, incorporated herein by reference).

Pegylation of several proteins has been shown to decrease their immunogenicity (see He, X.-H. et al. Reducing the immunogenicity and improving the in vivo activity of trichosanthin by site-directed pegylation. Life Sciences 65:355-368, 1999, and references cited therein, incorporated herein by reference). According to the present invention, site-directed pegylation of botulinum toxin will reduce its immunogenicity, thereby overcoming the development of antibody-mediated resistance to the toxin.

A commercially available pharmaceutical composition containing botulinum toxin is sold under the trademark BOTOX® (Allergan, Inc., Irvine, Calif.). It consists of a purified botulinum toxin type A complex, albumin and sodium chloride packaged in sterile, vacuum-dried form. The BOTOX® can be reconsistuted with sterile, non-preserved saline prior to intramuscular injection (which should preferably occur within four hours after reconstitution).

It has been reported that botulinum toxin type A has been used in clinical settings as follows: (1) about 75-125 units of BOTOX® per intramuscular injection (multiple muscles) to treat cervical dystonia; (2) 5-10 units of BOTOX® per intramuscular injection to treat glabellar lines (brow furrows) (5 units injected intramuscularly into the procerus muscle and 10 units injected intramuscularly into each corrugator supercilii muscle); (3) about 30-80 units of BOTOX® to treat constipation by intrasphincter injection of the puborectalis muscle; (4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid; (5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired); and (6) to treat upper limb spasticity following stroke by intramuscular injections of BOTOX® into five different upper limb flexor muscles, as follows: (a) flexor digitorum profundus: 7.5-30 units; (b) flexor digitorum sublimus: 7.5-30 units; (c) flexor carpi ulnaris: 10-40 units; (d) flexor carpi radialis: 15-60 units; (e) biceps brachii: 50-200 units. See U.S. Pat. No. 6,358,926 (col. 5, lines 18-48). One unit of botulinum toxin is defined as the LD₅₀ upon intraperitoneal injection into female Swiss Webster mice weighing 18-20 grams each, or about 50 picograms of botulinum toxin (purified neurotoxin complex).

The dose and mode of injection of pegylated botulinum toxin will be selected so as to treat effectively disorders of inappropriate muscle contraction while producing minimal weakness of surrounding muscle and systemic effects. The toxin may be formulated into a pharmaceutical composition (i.e., a composition suitable for pharmaceutical use in a subject, including an animal or human) by any acceptable means. See Remington's Pharmaceutical Sciences (Mack Publishing Co., Easton, 19th ed. 1995), incorporated herein by reference. Such pharmaceutical compositions typical comprise a therapeutically effective amount of the toxin (i.e., a dosage sufficient to produce a desired result).

What is claimed is:

1. A modified botulinum toxin comprising a botulinum toxin coupled to polyethylene glycol.

2. The modified botulinum toxin of claim 1, wherein said modified botulinum toxin comprises at least two polyethylene glycol chains.

3. The modified botulinum toxin of claim 1, wherein said botulinum toxin is selected from the group consisting of botulinum toxin A, botulinum toxin B, botulinum toxin C, botulinum toxin D, botulinum toxin E, botulinum toxin F, and botulinum toxin G.

4. The modified botulinum toxin of claim 3, wherein said botulinum toxin is botulinum toxin A.

5. The modified botulinum toxin of claim 3, wherein said botulinum toxin is botulinum toxin B.

6. A modified tetanus toxin comprising a tetanus toxin coupled to polyethylene glycol.

7. The modified tetanus toxin of claim 6, wherein said modified botulinum toxin comprises at least two polyethylene glycol chains.

8. A pharmaceutical composition comprising an effective amount of the modified botulinum toxin of claim 1.

9. The pharmaceutical composition of claim 8, wherein said botulinum toxin is botulinum toxin A, botulinum toxin B, botulinum toxin C, botulinum toxin D, botulinum toxin E, botulinum toxin F, and botulinum toxin G.

10. The pharmaceutical composition of claim 9, wherein said botulinum toxin is botulinum toxin A.

11. The pharmaceutical composition of claim9, wherein said botulinum toxin is botulinum toxin B.

12. A pharmaceutical composition comprising an effective amount of the modified tetanus toxin of claim 6.

13. A method of treating a subject suspected of having a disorder of inappropriate muscle contraction, wherein a therapeutically effective amount of the modified botulinum toxin of claim 1 is administered to the patient.

14. The method of claim 13, wherein said disorder of inappropriate muscle contraction is selected from the group consisting of low back pain, cervical dystonia, constipation, cerebral palsy, spastic paresis, blepharospasm, strabismus, hyperhydrosis, hypersialorrhoea, whiplash, migration headache and tension headache.

15. A method of treating a subject suspected of having a disorder of inappropriate muscle contraction, wherein a therapeutically effective amount of the modified tetanus toxin of claim 6 is administered to the patient.

16. A method of treating a patient for a cosmetic purpose, wherein an effective amount of a modified defined in claim 1 is administered to the patient.

17. The method of claim 16, wherein said cosmetic purpose is the reduction of facial wrinkles.
